(19) United States
(12) Patent Application Publication
(10) Pub. No.: US 2023/0227821 A1
(43) Pub. Date: Jul. 20, 2023

(54) COMPOSITIONS AND METHODS FOR ENHANCING ACTIVATION AND CYTOLYTIC ACTIVITY OF CD8+ T CELLS THROUGH DISRUPTION OF THE SAGA (SPT-ADA-GCN5-ACETYLTRANSFERASE) COMPLEX

(71) Applicant: UNIVERSITY HEALTH NETWORK, Toronto (CA)

(72) Inventors: Daniel DINIZ DE CARVALHO, Toronto (CA); Housheng HE, Toronto (CA); Ilias ETTAYEBI, Burlington (CA); Fraser SOARES, Toronto (CA); Helen LOO YAU, Toronto (CA)

(73) Assignee: UNIVERSITY HEALTH NETWORK, Toronto, ON (CA)

(21) Appl. No.: 17/916,981

(22) PCT Filed: Apr. 7, 2021
(86) PCT No.: PCT/CA2021/050464

§ 371 Date: Oct. 4, 2022

Related U.S. Application Data
(60) Provisional application No. 63/006,455, filed on Apr. 7, 2020.

(51) Int. Cl. C12N 15/113; C12N 5/0783; A61K 35/17; A61P 35/00

(52) U.S. Cl. CPC C12N 15/113; C12N 5/0636; A61K 35/17; A61P 35/00; C12N 2310/20

(57) ABSTRACT

Methods of increasing T cell effector function in a T cell population are provided that involve inhibiting one or more genetic subunits of the SAGA (Spt-Ada-Gcn5-acetyltransferase) gene regulation complex in the T cell population. Also provided are methods of using such T cell populations in the treatment of cancer patients.

This patent application claims priority to U.S. provisional patent application 63/006,455 filed Apr. 7, 2020.

### TECHNICAL FIELD

This application relates to methods of increasing the activation, proliferation or cytolytic activity of CD8⁺ T cells.

### BACKGROUND OF THE ART

### The SAGA Complex

The SAGA (Spt-Ada-Gcn5-acetyltransferase) complex is an evolutionary conserved, multifunctional co-activator comprising 19 subunits [1]. It is organized into separate modules with distinct activities, containing a structural core, a histone acetyltransferase (HAT), a histone deubiquitinase (DUB), and an activator-binding module [2]. SAGA and its related complexes are involved in several distinct signaling pathways, mostly through stimulating transcription via two chromatin-modifying enzymatic modules and by delivering the TATA box binding protein (TBP) to nucleate the pre-initiation complex on DNA, a pivotal event in the expression of protein-encoding genes [1].

Transcription of protein-encoding genes begins with the formation of a pre-initiation complex (PIC) comprising RNA polymerase II and several general transcription factors. In addition to SAGA, another multiprotein complex, transcription factor IID (TFIID), can deliver TBP to gene promoters, and is required for global gene expression in yeast. Recent studies have indicated a more specific role for SAGA than TFIID. For example, SAGA-dominated promoters tend to have consensus TATA box, are more stress-regulated/inducible genes, and tend to be more tightly regulated [3]. Upon deletion of SPT3, a TBP-interacting subunit of SAGA, Huisinga et al. showed that levels of total mRNA were reduced for only about 10% of yeast genes [4] compared to those regulated by TBP-associated factor 1 (Taf1), a subunit of the TFIID which reduced for roughly 90% of yeast genes. This led to the distinction between two different gene classes: (1) the SAGA-dominated genes, which are positively regulated by Spt3, but are essentially independent of Taf1, and (2) the larger class (90%) of TFIID-dominated genes, which are more dependent on Taf1 than on Spt3 [4]. Therefore, as a general model, it was proposed that TBP recruitment is primarily dependent on SAGA at TATA-containing promoters but dominated by TFIID at the TATA-like (or TATA-less) promoters (reviewed in [2]). Nonetheless, the central module has been shown to be structurally related to TFIID, suggesting that TBP binding in both complexes shares some common features.

A recent study by Baptista et al. observed a compensatory increase of the half-life of a majority of mRNAs upon SAGA depletion, explaining the limited changes in steady-state mRNA levels in the different SAGA mutant strains [5]. Therefore, the decrease in Pol II transcription following SAGA depletion was compensated by increasing mRNA half-lives, as previously reported for mutations in Pol II and Mediator, or inhibition of the kinase activity in TFIIH. These studies show the importance of SAGA in initiating transcription, and point to mechanisms of compensation when components of the SAGA complex are lost [5].

### CD8+ T Cell Activation

CD8+ T cells are cells of the adaptive immune system. Key features of the adaptive immune system are its ability to exercise immune responses against specific antigens (or foreign pathogens), and to retain memory of the antigen. CD8+ T cells are very important for immune defense not only for tumour surveillance, but they are also capable of delivering cytotoxic agents to kill infected cells, and tumour cells [6].

To become activated and perform its effector functions, naïve antigen specific CD8+ T cells require two signals for optimal activation:

### Signal 1—Antigen Recognition

This signal is delivered through antigen presentation by peptide: MHC class I complex antigen-bearing cell, with the T cell receptor (TCR) to ensure the specificity of the immune response. Although the TCR provides specificity, it does not possess the capacity for intracellular signalling. However, signal transduction is delivered through CD3 molecules that are associated with the TCR. CD3 molecules contain immunoreceptor tyrosine-based activation motifs (ITAMs) in their cytoplasmic tails in order to transduce signals. Ligation of the TCR with its cognate MHC class I results in phosphorylation of the ITAMs by Src family kinases, Lck and Fyn. Phosphorylated ITAMs serve as a docking site for the formation of a proximal signalling complex composed of Lck and Fyn, and the Syk family kinase called zeta-associated protein kinase 70 (ZAP-70). Downstream signalling through these pathways cause an intracellular rise in calcium ions, ultimately leading to the activation of the phosphatase calcineurin. Activated calcineurin promotes the dephosphorylation of members of the nuclear-factor of activated T-cells (NFAT) family of transcription factors. Ultimately, engagement of the TCR triggers downstream signalling cascades, ultimately converging into the activation of the transcription factors: NFkB, NFAT, AP1 into the nucleus. Altogether, they induce specific gene transcription programs, leading to cell proliferation and differentiation [7].

### Signal 2—Co-Stimulation

To support TCR signalling, a second co-stimulatory signal serves to promote the survival and expansion of T cells. The stereotypical co-stimulatory receptor found on T cells is CD28, expressed constitutively on the majority of naïve T-cells. Ligands of CD28 are CD80 and CD86 expressed on the surface of the antigen-presenting cell (APC). CD28 is found co-localized with the TCR in the central region of the immunological synapse, thus enhancing the events at the proximal signalling complex following TCR ligation. Thus, together with TCR signalling, CD28 can promote cytokine production, cellular cycling, survival, and differentiation [7,8].

CD8+ T cells also display a variety of receptors other than CD28 for co-stimulation signalling. Other receptor molecules, such as Programmed cell death protein 1 (PD-1) and cytotoxic T-lymphocyte antigen 4 (CTLA-4) can instead provide inhibitory signals, which can promote T-cell inhibition rather than activation [9]. Both co-stimulatory and co-inhibitory receptors have essential roles in T cell biology, as they determine the functional outcome of T cell receptor (TCR) signalling; also, important to maintain homeostasis [10].

### BRIEF SUMMARY

In one aspect, there is provided a method for increasing T cell effector function in a T cell population, the method comprising inhibiting the expression or function of the SAGA (Spt-Ada-Gcn5-acetyltransferase) gene regulation complex in T cells of the T cell population.

In one aspect, there is provided a method for increasing T cell effector function in a T cell population, the method comprising inhibiting one or more genetic subunits of the SAGA gene regulation complex in T cells of the T cell population. The one or more genetic subunits may be selected from ADA2B, CCDC101, TADA1, TAF5L, TAF6L, TAF10, SUPT7L, and TRRAP.

In one aspect, there is provided a method for increasing T cell effector function in a subject, the method comprising: contacting a T cell population with a composition comprising an inhibitor of the SAGA gene regulation complex ex vivo; and administering a therapeutically effective amount of the T cell population to the subject. In one embodiment, the subject is a patient diagnosed with cancer. In some embodiments the cancer is haematological. In some embodiments, the cancer is a leukaemia, lymphoma or myeloma. In some embodiments, the method further includes administering a cancer therapy to the subject, which may be an immunotherapy. In one embodiment, the method comprises administering to the subject an immune checkpoint inhibitor.

In all of the above methods, the T cells may be activated CD8⁺ T cells.

In various embodiments, the methods reduce the activity of the SAGA gene regulation complex in the T cells by at least 70%, at least 80%, at least 90% or at least 99%, relative to a population of T cells wherein the SAGA gene regulation complex is uninhibited.

In some embodiments of the above methods, the T cells express a Chimeric Antigen Receptor (CAR).

In various embodiments, the inhibitor may be selected from a small molecule; a nucleic acid capable of hybridizing with a nucleic acid encoding a genetic subunit of the SAGA gene regulation complex to inhibit the expression of the subunit; and a Cas9 protein or a polynucleotide encoding the Cas9 protein and a CRISPR-cas system guide RNA polynucleotide.

In another aspect, there is provided a modified T cell that expresses a Chimeric Antigen Receptor (CAR) and wherein the expression or function of one or more of the subunits of the SAGA gene regulation complex is inhibited. Also provided is a population of cells comprising a plurality of these modified T cells. In another aspect, there is provided a population of cells for use in a method of cancer treatment comprising a population of T cells, wherein one or more genetic subunits of the SAGA gene regulation complex are inhibited in the T cell population. The T cells of these populations of cells are suitably activated CD8⁺ T cells. The one or more genetic subunits may be selected from ADA2B, CCDC101, TADA1, TAF5L, TAF6L, TAF10, SUPT7L, and TRRAP. In various embodiments, the activity of the SAGA gene regulation complex in these populations of T cells is inhibited by at least 70%, at least 80%, at least 90% or at least 99%. In some embodiments, the inhibitor comprises a nucleic acid capable of hybridizing with a nucleic acid encoding a genetic subunit of the SAGA gene regulation complex to inhibit the expression of the subunit or a Cas9 protein or a polynucleotide encoding the Cas9 protein and a CRISPR-cas system guide RNA polynucleotide.

Disclosed embodiments include:
1. A composition for use in therapy comprising a population of T cells, wherein the expression or function of the SAGA (Spt-Ada-Gcn5-acetyltransferase) gene regulation complex has been inhibited in the T cell population.
2. A composition for use in therapy comprising a population of T cells, wherein one or more genetic subunits of the SAGA gene regulation complex are inhibited in the T cell population, preferably wherein the one or more genetic subunits are selected from ADA2B, CCDC101, TADA1, TAF5L, TAF6L, TAF10, SUPT7L, and TRRAP.
3. The composition of embodiment 2, wherein at least one of the one or more genetic subunits of the SAGA gene regulation complex is inhibited by a nucleic acid capable of hybridizing with a nucleic acid encoding the genetic subunit of the SAGA gene regulation complex to inhibit the expression of the subunit.
4. The composition of embodiment 2, wherein at least one of the one or more genetic subunits of the SAGA gene regulation complex is inhibited by a gene editing molecule, optionally a Cas9 protein or a polynucleotide encoding the Cas9 protein and a CRISPR-cas system guide RNA polynucleotide.
5. The composition of any one of embodiments 1 to 4, wherein the therapy is an allogeneic cell therapy.
6. The composition of any one of embodiments 1 to 5 wherein the T cells are activated CD8⁺ T cells.
7. The composition of any one of embodiments 1 to 6, wherein the activity of the SAGA gene regulation complex in the T cells is reduced by at least 70%, at least 80%, at least 90% or at least 99% relative to a population of T cells wherein the SAGA gene regulation complex is uninhibited.
8. The composition of any one of embodiments 1 to 7, wherein the T cell expresses a Chimeric Antigen Receptor (CAR).
9. The composition of any one of embodiments 1 to 8, further comprising an inhibitor of one or more subunits of the SAGA gene regulation complex.
10. The composition of embodiment 9, wherein the inhibitor is a small molecule.
11. The composition of any one of embodiments 1 to 10 for use in a method of treating cancer (e.g. leukaemia, lymphoma or myeloma).
12. The composition of any one of embodiments 1 to 11, wherein the therapy further comprises administering a cancer therapy to the subject.
13. The composition of embodiment 12, wherein the cancer therapy is an immunotherapy, preferably comprising administering to the subject an immune checkpoint inhibitor.
14. A method for increasing T cell effector function in a T cell population (preferably activated CD8⁺ T cells), the method comprising inhibiting the function and/or expression of the SAGA (Spt-Ada-Gcn5-acetyltransferase) gene regulation complex in T cells of the T cell population.
15. A method for increasing T cell effector function in a T cell population (preferably activated CD8+ T cells), comprising inhibiting expression of one or more genetic subunits of the SAGA gene regulation complex, preferably wherein the one or more genetic subunits are selected from ADA2B, CCDC101, TADA1, TAF5L, TAF6L, TAF10, SUPT7L, and TRRAP; optionally,
   wherein expression of at least one of the one or more genetic subunits of the SAGA gene regulation complex is inhibited by a nucleic acid capable of hybridizing with a nucleic acid encoding the genetic subunit of the SAGA gene regulation complex to inhibit the expression of the subunit; and/or
   wherein expression of at least one of the one or more genetic subunits of the SAGA gene regulation complex is inhibited by a gene editing molecule, optionally a Cas9 protein or a polynucleotide encoding the Cas9 protein and a CRISPR-cas9 system guide RNA polynucleotide; optionally,
   wherein at least some of the T cells of the T cell population comprise a nucleic acid encoding a CAR polypeptide or wherein the method comprises engineering the T cells to express a CAR polypeptide.

### BRIEF DESCRIPTION OF THE DRAWINGS

FIG. 1. Pooled CRISPR screen per EXAMPLE 1 identifies negative regulators of CD8+ T cell proliferation. A) Schematic of CRISPR screen timeline. B) Sorting strategy of carboxyfluorescein succinimidyl ester (CFSE) high (low proliferation) and CFSE low (high proliferation) cells after T cell receptor (TCR) stimulation. C) Volcano plot showing sgRNA targets enriched in CFSE high (low proliferating) population. D) GSEA of sgRNA targets enriched in CFSE high (low proliferating) population. Genes enriched are CD247, CD3D, CD3G, and LCK. E) Validation of top hit (IRF4). Western blot showing complete knockout of IRF4 with 2 sgRNAs against IRF4 gene. F) Validation of top hit (IRF4). Flow cytometry showing knockout of IRF4 with 2 sgRNAs against IRF4 gene. G) Validation of top hit (IRF4). CFSE staining of wildtype and IRF4 knockout T cells after TCR stimulation.

FIG. 2. Pooled CRISPR screen per EXAMPLE 2 identifies positive and negative regulators of CD8+ T cell activation. A) schematic of CRISPR screen timeline. B) Distribution of log2 fold-change (LFC) values of CD25 high over CD25 low cells for guides in the library. Bottom: LFC for all four sgRNAs targeting genes enriched in CD25 high cells (darker) and depleted genes (lighter), overlaid on gray gradient depicting the overall distribution. C) Volcano plot showing sgRNA targets enriched in CD25 high (high activation) and D) CD25 low (low activation) populations. E) Gene Ontology (GO) enrichment analysis of sgRNA targets enriched in CD25 high (high activation) and F) CD25 low (low activation) populations. G) STRING DP network analysis of Top hits. H) Correlation between top hits of CD25 and CFSE screen.

FIG. 3. Pooled CRISPR screen per EXAMPLE 3 identifies positive and negative regulators of CD8+ T cell degranulation. A) Schematic of CRISPR screen timeline. B) Distribution of log2 fold-change (LFC) values of CD107A high over CD107A low cells for guides in the library. Bottom: LFC for all four sgRNAs targeting genes enriched in CD107A high cells (darker) and depleted genes (lighter), overlaid on gray gradient depicting the overall distribution. C) Volcano plot showing sgRNA targets enriched in CD107A high (high activation) and D) CD107A low (low activation) populations. E) GO enrichment analysis of sgRNA targets enriched in CD107A high (high activation) and F) CD107A low (low activation) populations. G) STRING DP network analysis of Top hits. H) Correlation between top hits of CD107A and CFSE screen.

FIG. 4. Confirmation of lentiviral-RNP electroporation method used for CRISPR Screen. Effective at knockout of candidate gene target, CD45, in primary human CD8+ T cells. CD45 control with no Cas9 showed that knockout is specific to cells transduced with the CD45 targeting guide complexed with Cas9 48 hrs after transduction.

FIG. 5. Summary of SAGA CRISPR screen results. A) SAGA complex components found in CRISPR screen. B) Targets overlapping between All CRIPSR screens. C) LFC for all sgRNAs targeting components of the SAGA complex (coloured lines correspond with modules of SAGA complex) in CFSE CRISPR screen, overlaid on gray gradient depicting the overall distribution. Top ranked targets are TADA2B, TAF6L, TADA1. D) LFC for all sgRNAs targeting components of the SAGA complex (coloured lines correspond with modules of SAGA complex) in CD25 CRISPR screen, overlaid on gray gradient depicting the overall distribution. Top ranked targets are TADA2B, SUPT7L, TAF10, TAF6L, TADA1 E) LFC for all sgRNAs targeting components of the SAGA complex (coloured lines correspond with modules of SAGA complex) in CD107A CRISPR screen, overlaid on gray gradient depicting the overall distribution. Top ranked targets are SGF29 (orCCDC101), TADA3, TADA2B, SUPT7L, TAF10, TAF6L, TADA1, TAD5L, TRRAP, USP22.

FIG. 6. Validation of CD25 CRISPR screen I. A) Sorting strategy of CD25 high (low activation) and CD25 low (high activation) cells after TCR stimulation. B) Volcano plot showing CD25 (IL2RA) as the top hit in CD25 low population. C) Validation of IRF4 knockout CD8 T cells decreasing CD25 expression after TCR stimulation. D) Initial validation of components of SAGA complex increasing CD25 expression (and MFI) after TCR stimulation, measured at day 7 post SAGA target knockout. E) Schematic of optimized timeline for SAGA target validation to measure CD25, CD69, and HLA-DR on CD8 T cells. F) Fold change expression of CD25, CD69, and HLA-DR in CD8 T cells with SAGA targets knockout relative to control (N=2, two different biological samples, mean±SEM). At resting state and res-stimulated for 24 hours.

FIG. 7. Validation of CD107A CRISPR screen. A) Sorting strategy of CD107A high (low activation) and CD107A low (high activation) cells after TCR stimulation. B) Gating strategy for initial validation of components of SAGA complex increasing CD107A expression after TCR stimulation, measured at day 5 post target knock. C) Schematic of optimized timeline for SAGA target validation to measure CD107a, Granzyme B (GZMB) and Perforin (PRF1) on CD8 T cells. D) Fold change expression of CD107a, Granzyme B (GZMB) and Perforin (PRF1) in CD8 T cells with SAGA targets knockout relative to control (N=2, two different biological samples, mean±SEM).

FIG. 8. Validation of target killing potential by antigen specific CD8 T cells multiplexed with SAGA target knock out.

A) Luciferase luminescence measurements of Target cell killing by CD8+ T cells with USP22 gene knockout (SAGA target) and CBLB gene knockout (positive control) (N=3, single biological sample, mean±SEM).

B) Functional validation via Western blot depicting increased levels H2BK120-ub upon USP22 gene knockout in CD8+ T cells (N=2, two different biological samples).

### DETAILED DESCRIPTION

In cancer, antigen specific CD8+ cells become activated by tumour antigens, proliferate, and gain effector functions to exert anti-tumorigenic effects on cancer cells. Tumour-infiltrating lymphocytes (TILs) with cytotoxic phenotypes are found in various cancer tissue from patients and correlate to a better survival, regardless of the type of therapy administered [11]. Effector cytokines mediate these anti-tumour effects and are important for immunosurveillance and tumour elimination.

The role of SAGA complex in immune cells is not well studied. Recently, there have been some studies characterizing certain modules of the SAGA complex. For example, Zhang et al. found that one of the components of the histone deubiquitinase (DUB) module of the SAGA complex, ubiquitin-specific peptidase 22 (USP22) was highly expressed in iNKT cells during their early developmental stage 1 and that USP22 deficiency blocked the transition from stage 1 to 2 during iNKT cell development in a cell intrinsic manner [12]. Mechanistically, USP22 interacted with the Mediator complex subunit 1 (MED1), a transcription coactivator involved in iNKT cell development, enhancing MED1 function for IL-2Rβ and T-bet gene expression through deubiquitinating histone H2A monoubiquitination. More recently, reports have eluded to a role of the SAGA complex in mouse regulatory T cell function.

Using a genome wide pooled CRISPR screen, San Loo et al. identified various components of the SAGA complex, including Ccdc101, Tada2b, and Tada3 in the HAT module, Usp22 in the DUB module, and Tada1, Taf6I, Supt5, and Supt20 from the core structural module as positive Foxp3 regulators [13]. Similarly, Cortez et al. found similar SAGA modulators of Foxp3 including Usp22, Atxn7I3 [14]. These results point to an important role of the SAGA complex in modulating regulatory immune cells and their functions, however the role of SAGA in other T cell populations and the mechanism of how this occurs has not been established.

Genetic subunits of the human SAGA complex and their sequences are known in the art see e.g. the relevant entry of the HUGO Gene Nomenclature Committee, and include ataxin 7 (ATXN7); ataxin 7 like 3 (ATXN7L3); ENY2 transcription and report complex 2 subunit (ENY2); lysine acetyltransferase 2A (KAT2A); lysine acetyltransferase 2B (KAT2B); SAGA complex associated factor 29 (SGF29); SPT3 homolog, SAGA and STAGA complex component (SUPT3H); SPT7 like, STAGA complex subunit gamma (SUPT7L); SPT20 homolog, SAGA complex component (SUPT20H); transcriptional adaptor 1 (TADA1); transcriptional adaptor 2B (TADA2B); transcriptional adaptor 3 (TADA3); TATA-box binding protein associated factor 5 like (TAF5L); TATA-box binding protein associated factor 6 like (TAF6L); TATA-box binding protein associated factor 10 (TAF10); TATA-box binding protein associated factor 12 (TAF12); transformation/transcription domain associated protein (TRRAP); ubiquitin specific peptidase 22 (USP22). The amino acid and encoding nucleotide sequences are also available to those of skill in the art through the National Center for Biotechnology Information (NCBI) CODS Database, available through the NCBI Website (https://www.ncbi.nlm.nih.gov).

In some embodiments, the method involves inhibiting one or more of the genetic subunits TADA2B (ADA2B), SGF29 (CCDC101), TADA1, TAF5L, TAF6L, TAF10, SUPT7L, and TRRAP.

The Examples that follow provide evidence that the SAGA complex plays an important role in modulating human CD8+ T cell proliferation, activation, and cytolytic potential post TCR stimulation. Knockout of various components of the SAGA complex, including the structural core, the histone acetyltransferase (HAT), the histone deubiquitinase (DUB), and the activator-binding module led to increased CD8+ T cell proliferation, activation, and active degranulation.

As used herein, “increasing T cell effector function” refers to enhancing the ability of a T cell population to recognize and/or have increased cytotoxic effect against a target cell, which may include increasing the proliferation of a T cell population, increasing the expression and/or excretion of cytotoxic molecules (e.g. perforin, granzymes, granulysin, Fas ligand) or cytokines e.g. TNF-α and IFN-γ) by a T cell population.

The term “subject” as used herein includes all members of the animal kingdom including mammals, and, in particular, includes humans.

In some embodiments, the subject has been diagnosed with cancer.

The term “cancer”, as used herein, may mean a malignant neoplasm that has undergone characteristic anaplasia with loss of differentiation, increased rate of growth, invasion of surrounding tissue, and is capable of metastasis. Metastatic cancer is a cancer at one or more sites in the body other than the site of origin of the original (primary) cancer from which the metastatic cancer is derived.

The term “tumor”, as used herein, refers to a neoplasm or an abnormal mass of tissue that is not inflammatory, which arises from cells of pre-existent tissue. A tumor can be either benign (noncancerous) or malignant (cancerous). Tumors can be solid or hematological.

Examples of hematological tumors include, but are not limited to: leukemias, lymphoma, myelomas. Examples of solid tumors include sarcomas and carcinomas.

Methods of increasing T cell effector function as provided herein may be broadly applicable to cancer patients diagnosed with various forms of cancer.

In some embodiments, the subject has been diagnosed with a bladder cancer, brain cancer, breast cancer, cervical cancer, colorectal cancer, esophageal cancer, head and neck cancer, kidney cancer, leukemia, liver cancer, lung cancer, lymphoma, melanoma, multiple myeloma, ovarian cancer, pancreatic cancer or prostate cancer.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

In one embodiment, there is provided a method for increasing T cell effector function in a T cell population, the method comprising inhibiting one or more genetic subunits of the SAGA gene regulation complex in T cells of the T cell population

The term “immunotherapy” as used herein refers to methods and compositions that induce or enhance the targeting or destruction of cancer cells by the immune system. Immunotherapies include immune checkpoint inhibitors, monoclonal antibodies, T-cell therapy (e.g. CAR-T), oncolytic virus therapy, and cancer vaccines.

Known immune checkpoint inhibitors include, but are not limited to ipilimumab, pembrolizumab, nivolumab, atezolizumab, avelumab, and durvalumab.

Immune checkpoint blockade (ICB) has led to improved clinical outcomes in several types of cancer. However, the majority of patients treated with ICB fail to respond, leading to overall response rates of 20% to 40%. Preclinical studies have demonstrated that the relative abundance of suppressive cells versus cytotoxic T cells determines the efficacy of combination immunotherapies. In addition, the abundance of tumor-infiltrating T cells is a major factor predicting response to immunotherapy, as T-cell inflamed tumors are more sensitive to ICB than non-T-cell inflamed tumors. Strategies of regulating the immune tumor microenvironment (TME) is therefore a promising therapeutic opportunity that can be leveraged to improve response rates to immunotherapy.

Clinical applications of the methods provided herein include the field of adoptive cell therapy (e.g. Tumor-Infiltrating Lymphocyte (TIL) Therapy, Engineered T Cell Receptor (TCR) Therapy and Chimeric Antigen Receptor (CAR) T Cell Therapy), where these targets can be inhibited on human primary CD8+ T cells ex vivo, and the CD8+ T cells re-introduced back into patients as a means of improving overall response rates of patients with cancer. Cell therapies may be autologous or allogeneic.

The term “chimeric antigen receptors (CARs)” as used herein refers to T-cell receptors engineered to graft an artificial specificity onto the immune cell. The CARs may be employed to impart the specificity of a monoclonal antibody onto a T cell for use e.g. in adoptive cell therapies. In specific embodiments, the CARs direct specificity of the cell to a tumor associated antigen. In some embodiments, the CARs comprise an intracellular activation domain, a transmembrane domain and an extracellular domain comprising a tumor associated antigen binding region.

Methods of inhibiting these targets include by small molecules, genetic engineering such as CRISPR knockout or RNA interference knockdown.

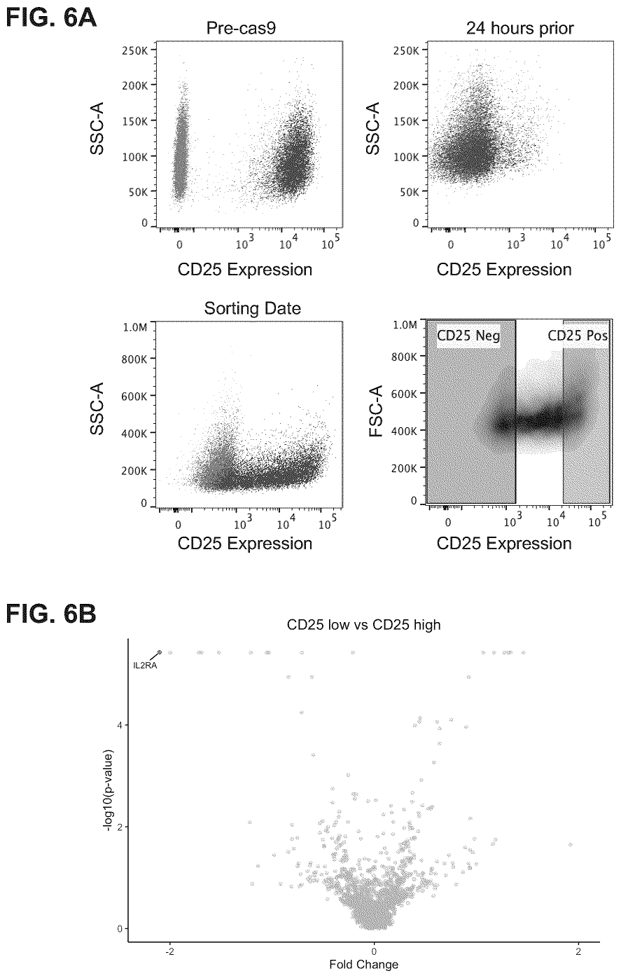

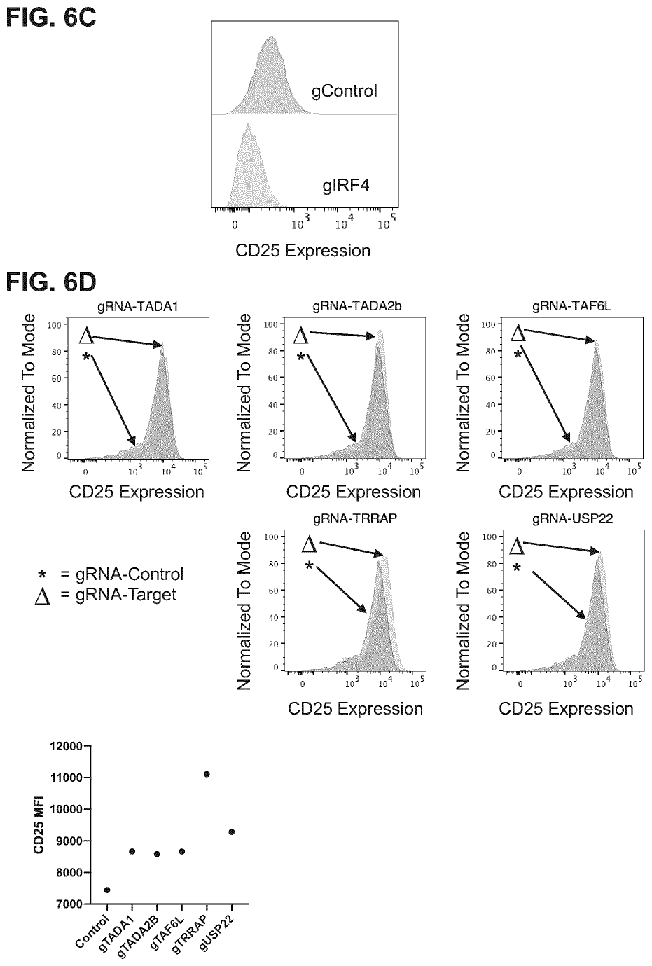

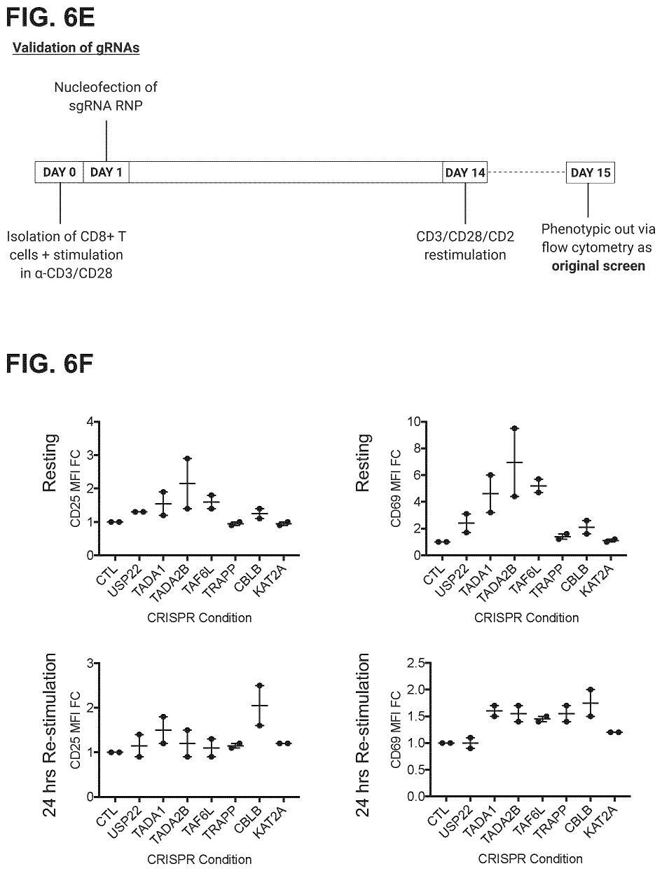

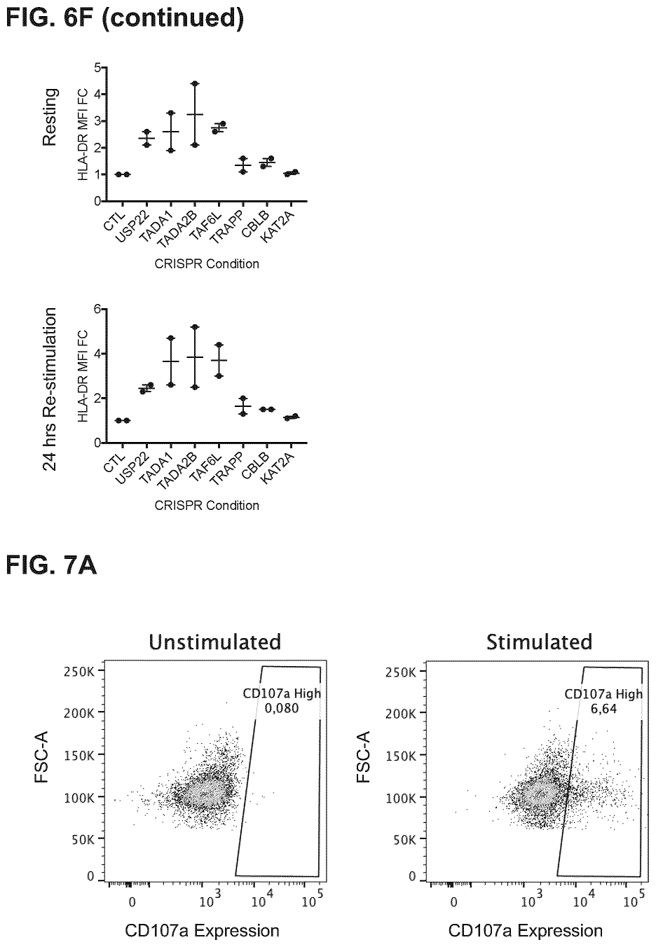

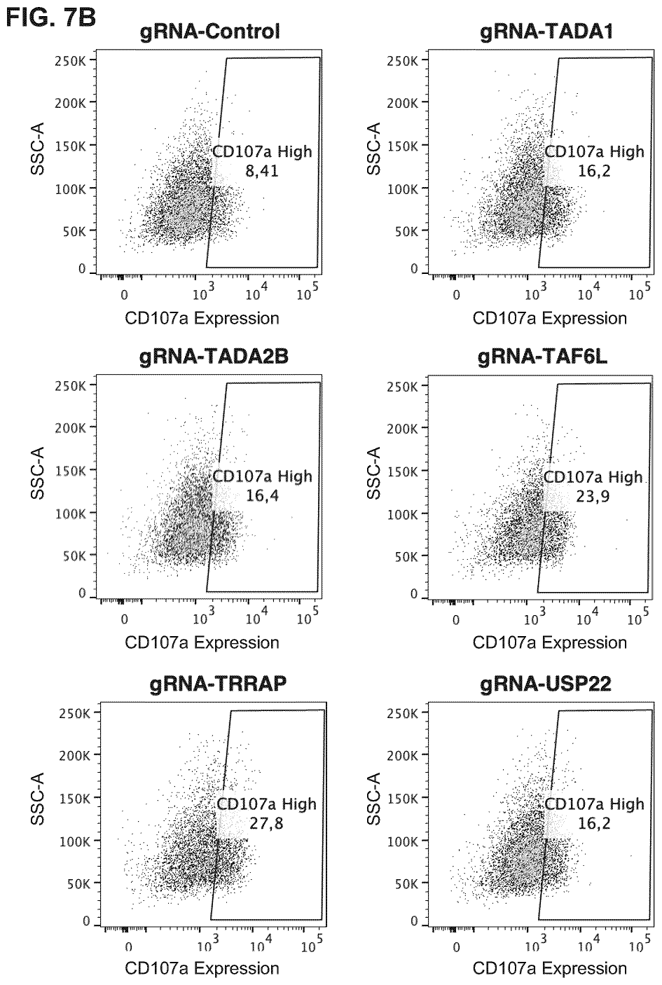

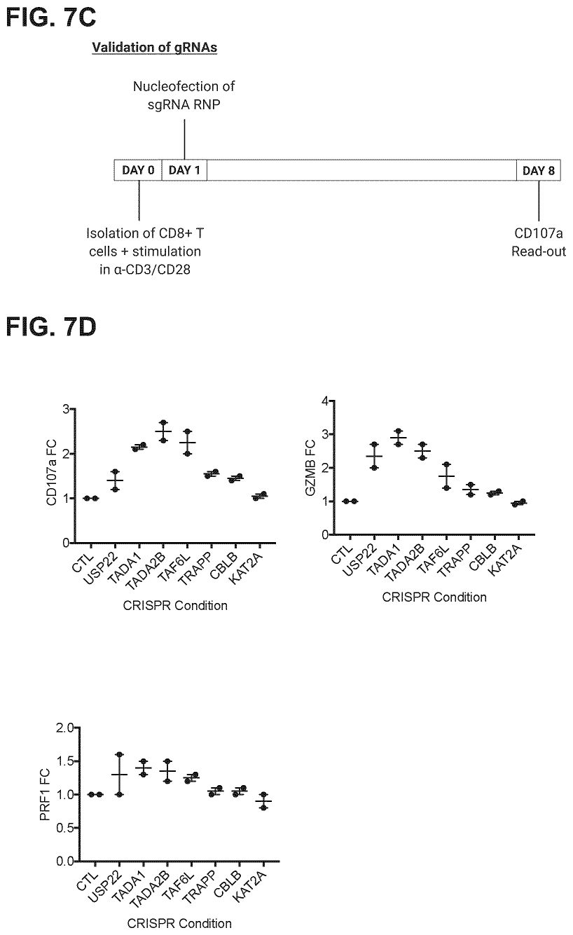

Small molecule inhibitors for use in the methods provided herein include, but are not limited to, GSK4027 and L-Moses inhibitor (inhibitors of KAT2A). Broad spectrum histone deacetylase inhibitor, trichostatin A, and antineoplastic agent, pirarubicin, have been reported to affect USP22 expression in cancer cells (PMID: 31007754, PMID: 25323692). Methods of confirming the activity of small molecule inhibitors will be known to persons of skill in the art and further are described herein. In particular, the methodology shown in FIGS. 6 and 7 and detailed in the examples may be followed.

RNA interference is used to modulate the expression of a target genes by employing small ribonucleic acid molecules that are present in duplex structures. Cytosolic delivery of siRNA oligonucleotides or viral integration of shRNA leads to transient and stable downregulation of gene expression respectively. The mechanism of RNAi is based on the sequence-specific degradation of host mRNA through the cytoplasmic delivery of double-stranded RNA (dsRNA) identical to the target sequence [15]. Degradation of target gene expression is achieved through an enzymatic pathway involving the endogenous RNA-induced silencing complex (RISC). One strand of the siRNA duplex (the guide strand) is loaded into the RISC with the assistance of Argonaute (AGO) proteins and double-stranded RNA-binding proteins. The RISC then localizes the guide strand to the complementary mRNA molecule, which is subsequently cleaved by AGO near the middle of the hybrid [16]. Alternatively, the programmable RNA targeting enzyme, Cas13d, can be employed to manipulate expression of target genes by utilizing a CRISPR RNA (cRNA) [17]. Cas13d is guided to RNA by the cRNA that contains a complementary spacer sequence (guide) found on host mRNA. Degradation of host mRNA occurs by Cas13d mediated cleavage of the RNA-RNA hybridization.

In some embodiments, any suitable gene editing technology may be used to inhibit the expression of the SAGA complex in a T cell/population of T cells. In some embodiments, a CRISPR-Cas system may be used to inhibit expression of one or more subunits of the SAGA complex in a population of T cells. In one embodiment, these T cells are obtained from the patient. As will be known to person of skill in the art, CRISPR systems involves a guide sequence which is designed to have complementarity to target nucleotide sequence within a cell. Hybridization between a target sequence and a guide sequence promotes the formation of a CRISPR complex of a guide sequence hybridized to a target sequence and complexed with one or more Cas proteins, which in turn causes cleavage of one or both strands of the polynucleotide in or near the target sequence. Vector systems for expression of the functional components needed for CRISPR-Cas genome editing are also known to those of skill in the art and can be purchased from commercial sources. Further description of CRISPR-Cas systems, including their application in eukaryotic cells, can be found in WO2014/204727. Further description of the use of CRISPR-Cas systems, including within murine and human T-cells can be found e.g. in Choi et al., Henriksson et al., Shifrut et al., and Su et al., [18, 19, 20, 21], incorporated herein by reference.

RNPs are used to knock out genes in primary T cells. RNPs are produced by complexing a two-component sgRNA to Cas9, as previously described. In brief, crRNAs and tracrRNAs are chemically synthesized, and recombinant Cas9-NLS, D10A-NLS, are recombinantly produced and purified. RNPs are electroporated 2 days after initial T cell activation with anti-CD3/CD28 antibodies, and maintained in media culture [22].

Suitable vectors will be known to persons of skill in the art and vectors are available from commercial sources and include plasmid and viral vectors (e.g. lentiviral vectors, adenovirus vectors, adeno-associated virus (AAV) vectors).

Delivery vehicles for nucleic acids are known to those of skill in the art, and include liposomes, lipoplexes or lipid nanoparticles.

Methods of assessing and quantifying the reduction in the activity of the SAGA gene regulation complex in a T cell population will be known to those of skill in the art.

Suitably, T cells isolated from healthy donors are subjected to electroporation with individual Cas9 ribonucleoproteins (RNPs) to achieve single-target-gene knockout in the T cells. The single-target-gene knockout include one of the genetic subunits TADA2B (ADA2B), SGF29 (CCDC101), TADA1, TAF5L, TAF6L, TAF10, SUPT7L, and TRRAP. Efficiency and level of knockout may be assessed through western blotting and flow cytometry (where possible) in order to measure protein level of these targets compared to wild type control. SAGA activity is evaluated by measuring global H2B ubiquitination and H3 acetylation levels by western blot. In some embodiments, the activity of the SAGA gene regulation complex can be decreased by at least 70%, at least 80%, at least 90% or at least 99% in a T cell population treated according to methods described herein. Single-target-gene knockout of these targets positively regulate both CD25 and CD107a expression, and overall T cell cytolytic capacity. The expression of various extracellular T cell activation markers, as well as, intracellular expression and secretion of cytolytic proteins and cytokines from edited T cells may be measured.

In some embodiments, T cells are activated with anti-CD3/CD28 antibodies prior to electroporation with single-target-gene knockout. T cells are expanded and maintained in culture media containing IL-2, IL-7 and IL-15 cytokines. This is synonymous to the process of how engineered antigen-specific T cells are manufactured [23] prior to infusion back into patients.

A recent study by Stadtmauer et al., demonstrated that multiplex human genome engineering is safe and feasible using CRISPR-Cas9 [23]. In this study, autologous T cells were CRISPR knockout with Cas9 ribonucleoproteins (RNPs) targeting the gene sequences TRAC, TRBC and PDCD1. T cells were cultured in media supplemented with IL-7 and IL-15. After 2 days in culture, these T cells are activated and expanded using anti-CD3/CD28 antibody-conjugated paramagnetic microbeads (Life Technologies). The next day, T cells are transduced with a lentiviral vector expressing the HLA-A*0201-restricted NY-ESO-1 (SLLMWITQC)-specific TCR. The strategy allowed for the manufacturing of NY-ESO-1 TCR-expressing engineered T cells, devoid of endogenous expression of alpha and beta TCR domain genes (by deleting TRAC and TRBC, respectively) and PD1 protein (by deleting PDCD1).

The examples evidence that disrupting the SAGA complex by genetic editing (such as CRISPR) or pharmacologically (small molecules inhibitors or degraders) increases T cell effector function (which may include increasing the proliferation of a T cell population, increasing the expression and/or excretion of cytotoxic molecules or cytokines) and this invention may be used in combination with other cell therapy approaches available: TILs, Engineered T Cell Receptor (TCR) Therapy, CAR-T cells, etc. These therapy approaches focus on increasing the number of T cells (expansion) and induce specificity towards cancer targets or cancer antigens, but they do not focus on enhancing the potential for these cells to kill target cancer cells (killing potential). Provided herein is a novel method to enhance the killing potential of these immune cell therapies by disrupting the SAGA complex.

Methods and protocols for administering cancer cell therapy are known to persons of skill in the art.

In some embodiments, cancer patients are further administered a cancer therapy. Cancer therapies are known in the art and include, but are not limited to, surgery, chemotherapy, radiation therapy, bone marrow transplants, immunotherapy, and hormone therapy. In one embodiment, a cancer patient is administered an immunotherapy treatment.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

The advantages of the present invention are further illustrated by the following examples. The examples and their particular details set forth herein are presented for illustration only and should not be construed as a limitation on the claims of the present invention.

### EXAMPLES

### Example 1

### Pooled CRISPR Screen Reveals Negative Regulators of Human CD8+ T Cell Proliferation

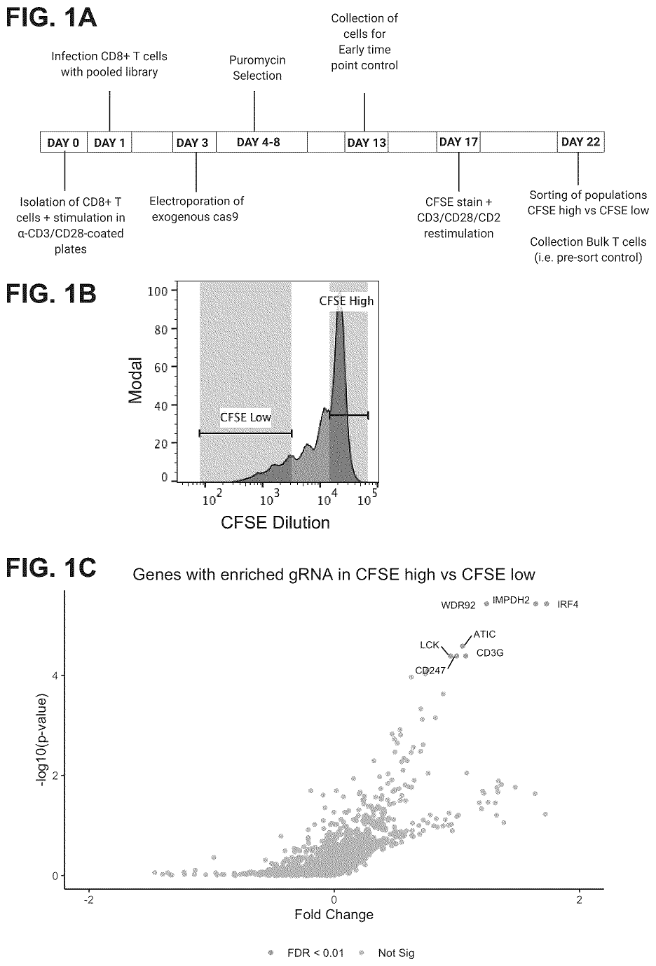

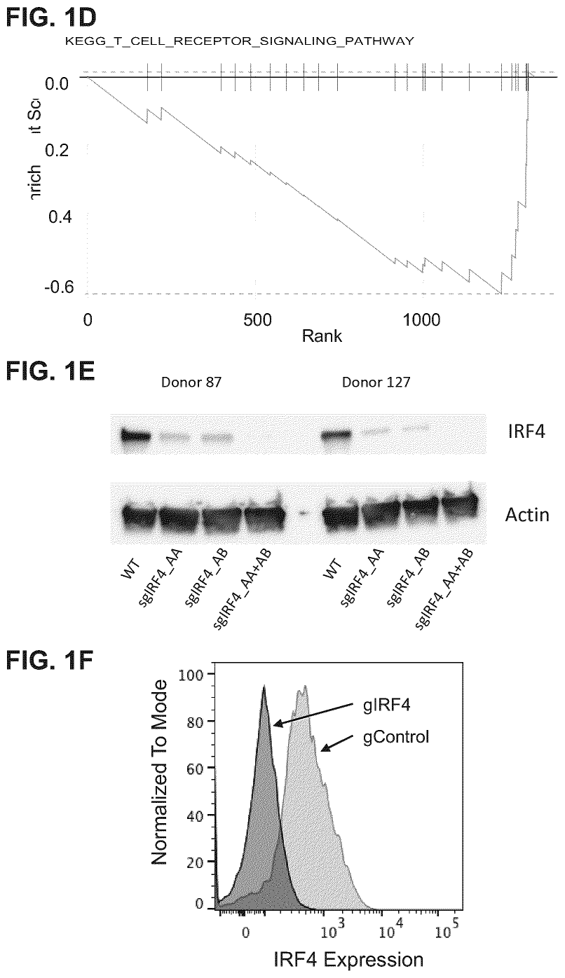

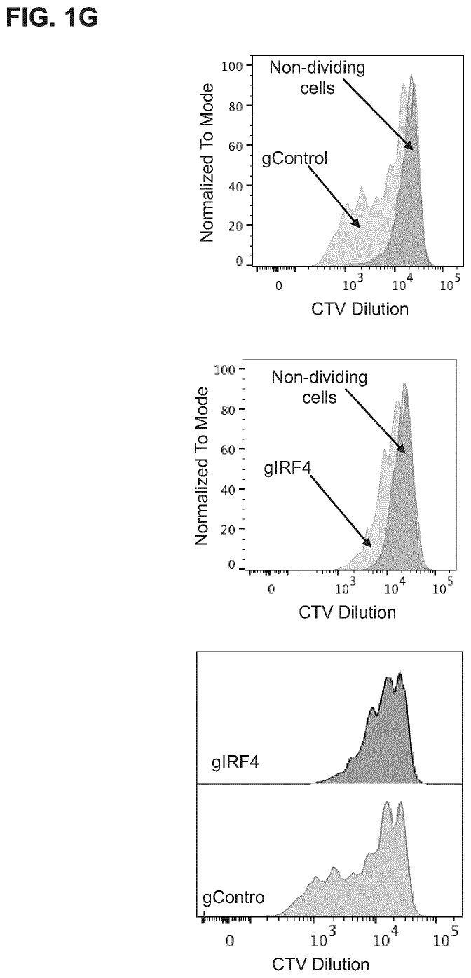

Using a pooled CRISPR screen, the present inventors sought to identify positive and negative regulators of human CD8+ T cells and their functional activity. As a first step, a screen was performed to identify gene targets that regulate T cell proliferation in response to T cell receptor (TCR) stimulation. The inventors devised a custom Epi-Drug CRISPR library of sgRNA plasmids targeting 657 genes with FDA approved drugs (with known pharmacological activity), 334 epigenetic regulators (many are targetable), and several canonical members of the TCR signaling pathway. With reference to FIG. 1A, CD8+ T cells isolated from a healthy human donor were transduced with lentivirus encoding this sgRNA library, electroporated with Cas9, maintained in culture for 10 days post-electroporation, labeled with carboxyfluorescein succinimidyl ester (CFSE) to track cell divisions and then TCR stimulated. With reference to FIG. 1B, cells were sorted by FACS into two populations: non-proliferating cells (CFSE high), and highly proliferating cells (CFSE low). With reference to the results shown in FIGS. 1C-1G: As expected, sgRNAs targeting essential components of TCR signaling, such as CD3G, LCK, and CD247 inhibited cell proliferation. Genes identified in the CFSE high population were enriched with annotated pathways associated with TCR stimulation. Gene set enrichment analysis (GSEA) revealed significant over-representation of gene targets depleted from proliferating cells in the TCR signaling pathway. In addition, sgRNAs targeting non-essential components of TCR signaling, but that have been previously shown to be important in T cell proliferation were found, such as IRF4. The top-ranking negative regulator was validated by individual CRISPR knockout with Cas9 ribonucleoproteins (RNPs). Taken together, these results show strong concordance of the targeted pooled CRISPR screen with previous proliferation based CRISPR screens and can be used to discover positive and negative regulators of primary human T cell activation and function.

### Example 2

### Pooled CRISPR Screen Reveals Positive and Negative Regulators of Human CD8+ T Cell Activation

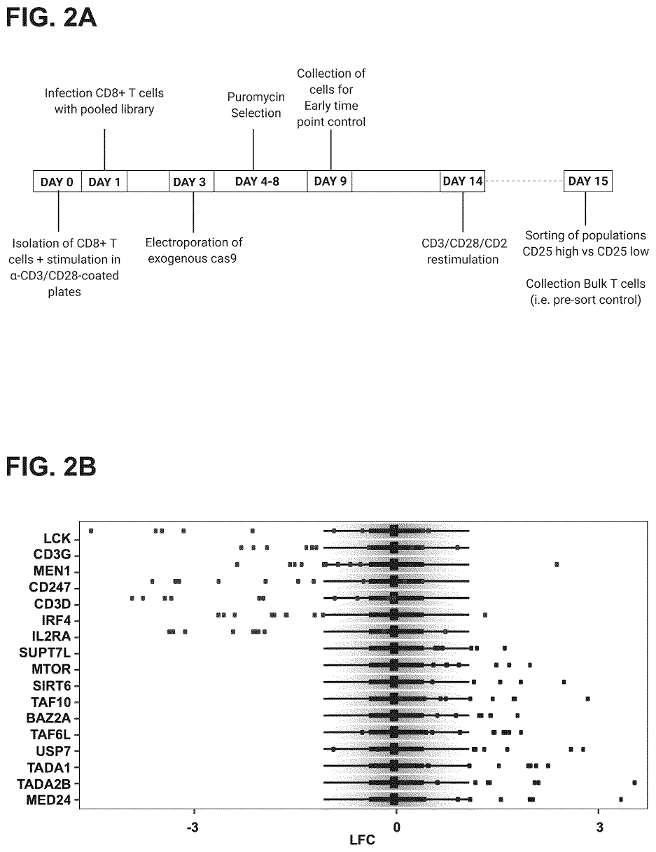

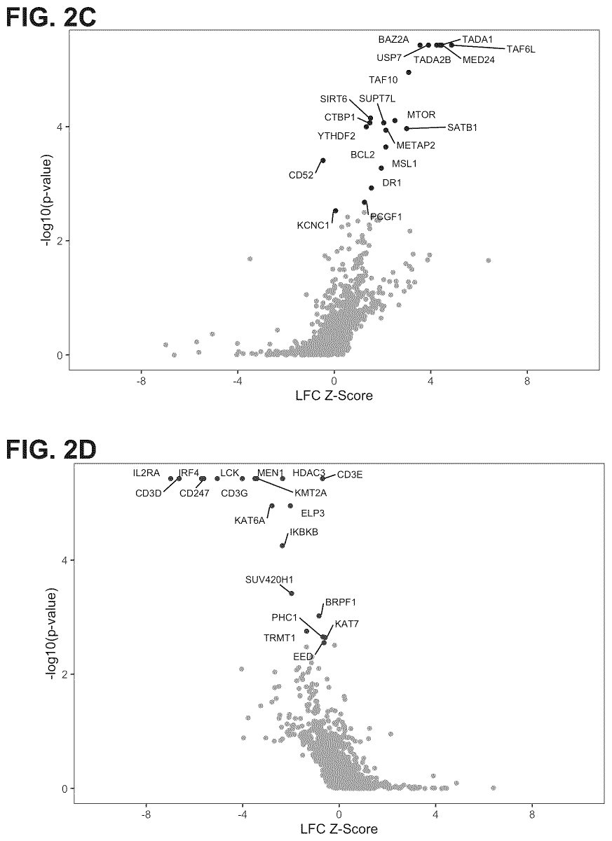

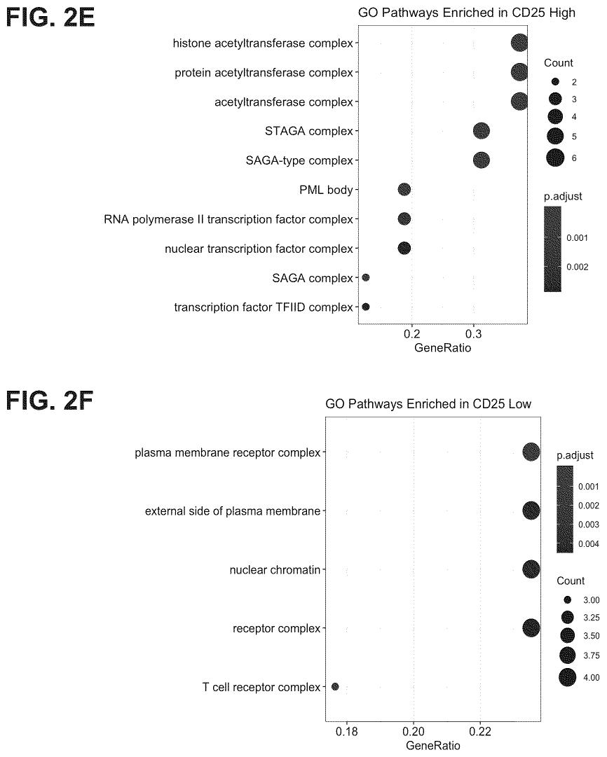

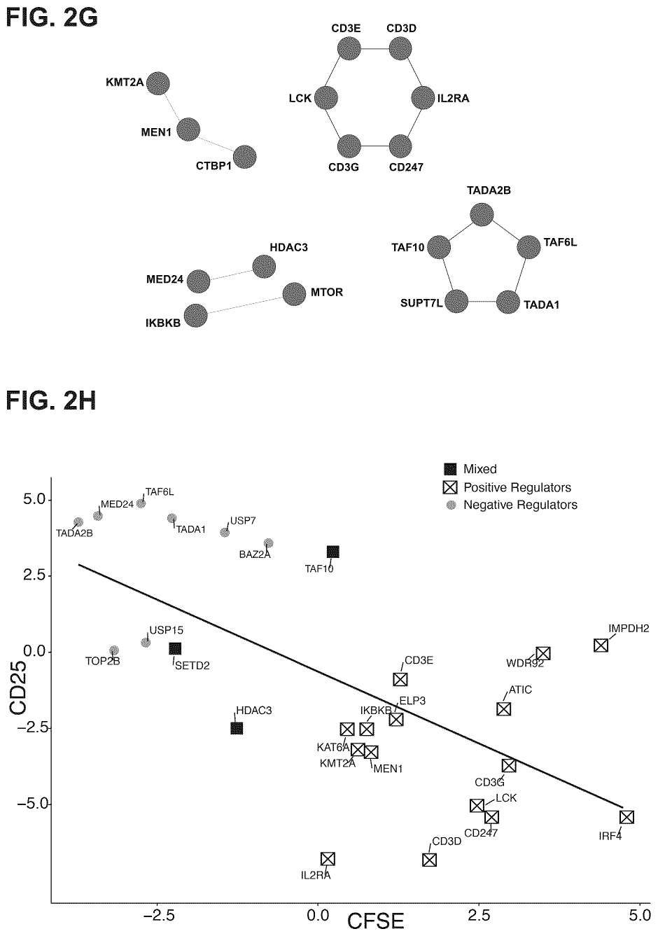

In order to investigate regulators of T cell activation, human CD8+ T cells were stimulated and a CRISPR screen sorting for the T cell activation marker CD25 (IL2RA) was performed (schematic of CRISPR screen is shown in FIG. 2A). Cells were sorted by FACS into two populations; highly activated cells (CD25 high), and lowly activated cells (CD25 low). With reference to the results shown in FIGS. 2B-2H: As expected, sgRNAs targeting established regulators of T cell activation including CD25 (IL2RA) itself, CD3D, LCK, CD247 and IRF4 were identified. IRF4 was validated by individual CRISPR knockout with Cas9 ribonucleoproteins (RNPs) and a strong decrease is CD25 expression was observed after IRF4 knockout. sgRNAs enriched in the highly activated T cells (CD25 high) were then investigated. Interestingly, various components of the SAGA (Spt-Ada-Gcn5-acetyltransferase) complex were identified including TADA2B (HAT module) and TADA1, TAF6L, TAF10 (core structure module) among the negative regulators of CD8+ T cell activation. In addition, Gene Ontology analysis revealed significant over-representation of gene targets in histone acetyltransferase and SAGA complex in T cells with high CD25 expression. Taken together, these data are in agreement with previous literature regarding positive regulators of CD8+ T cell activation and demonstrate an unappreciated role that the SAGA complex plays in modulating CD8+ T cell activation.

### Example 3

### Pooled CRISPR Screen Reveals Positive and Negative Regulators of Human CD8+ T Cell Cytolytic Activity

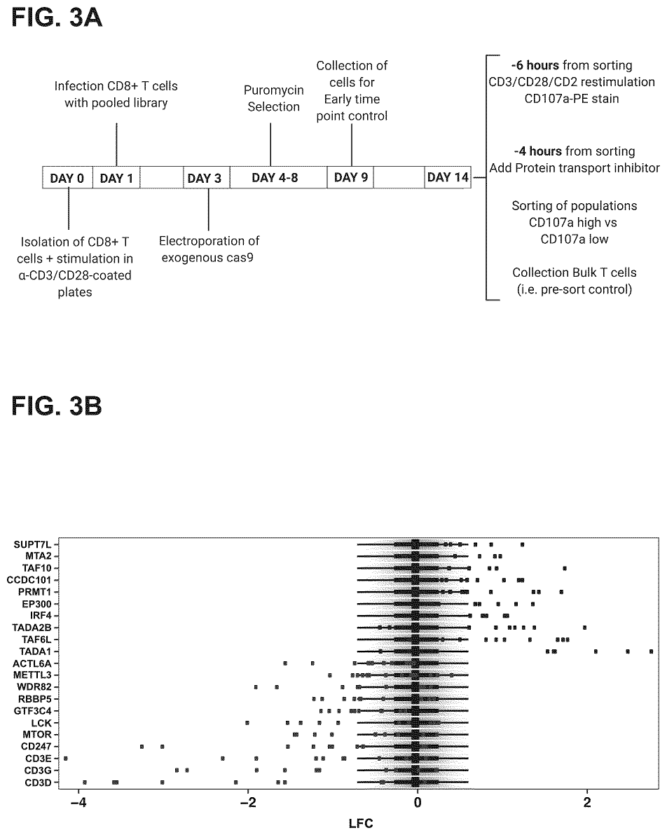

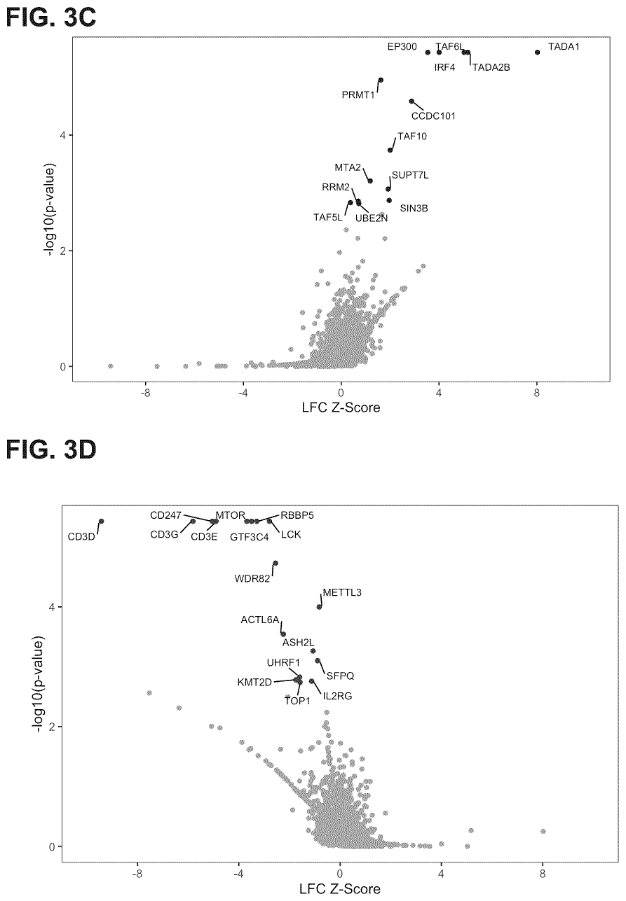

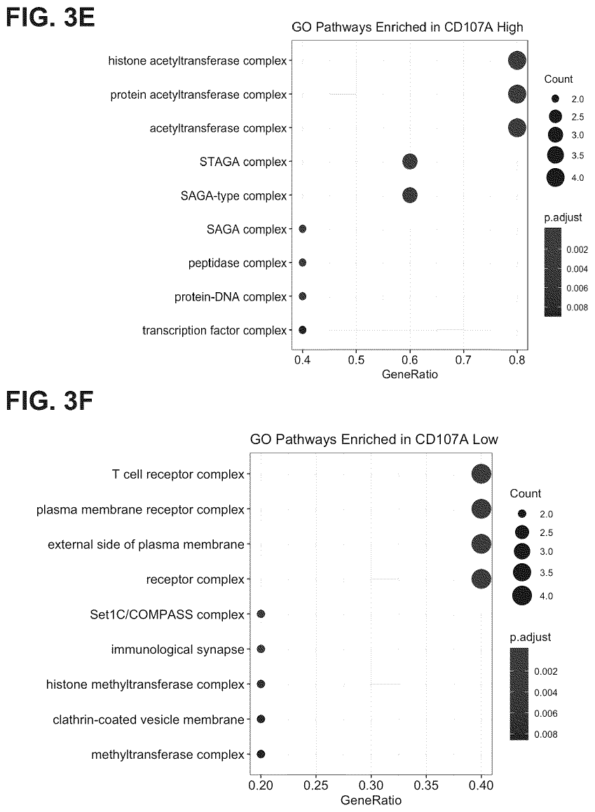

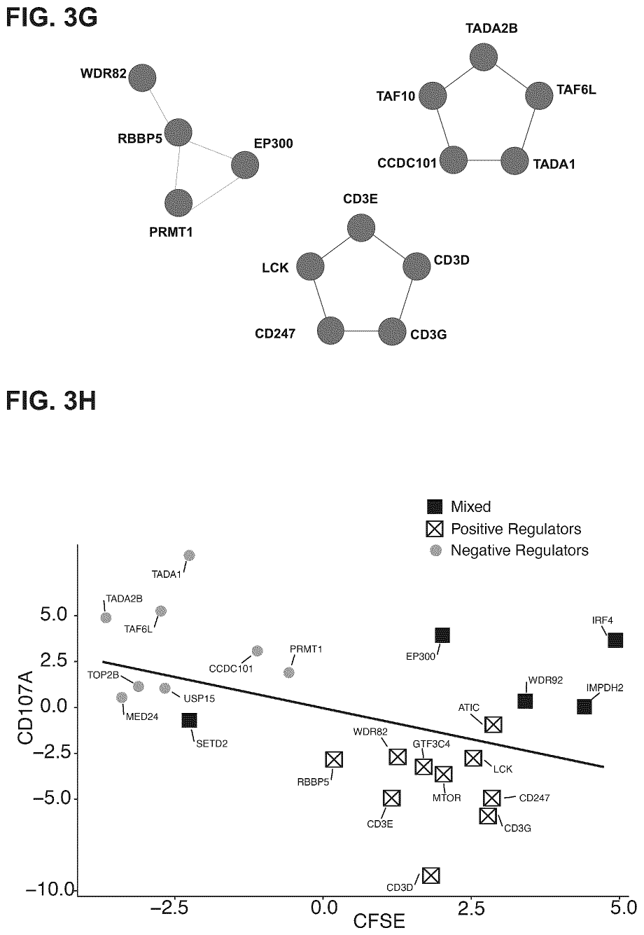

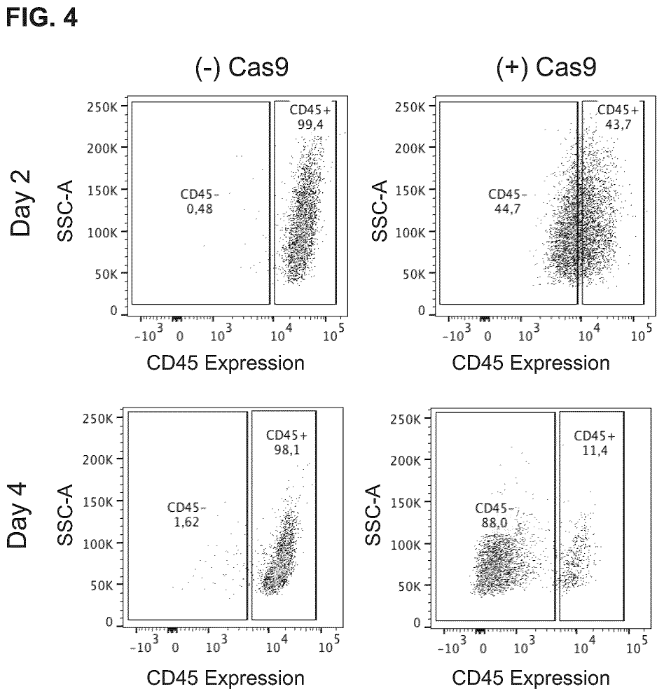

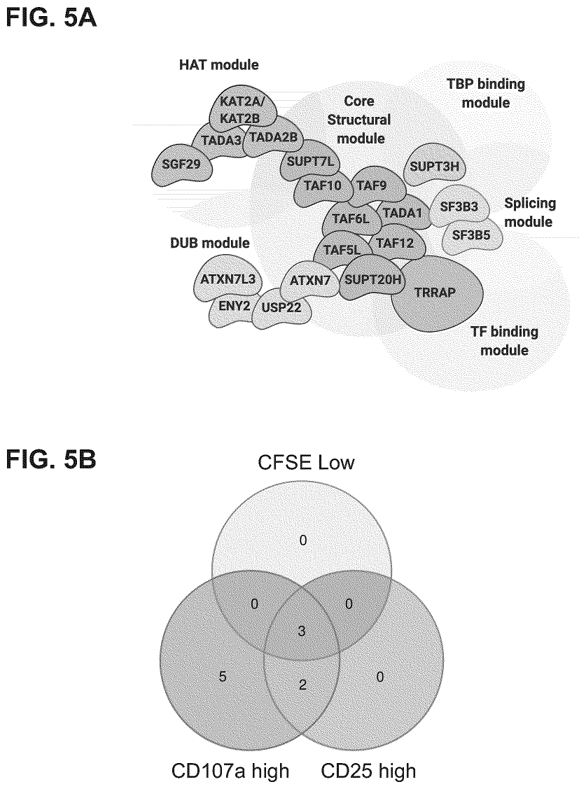

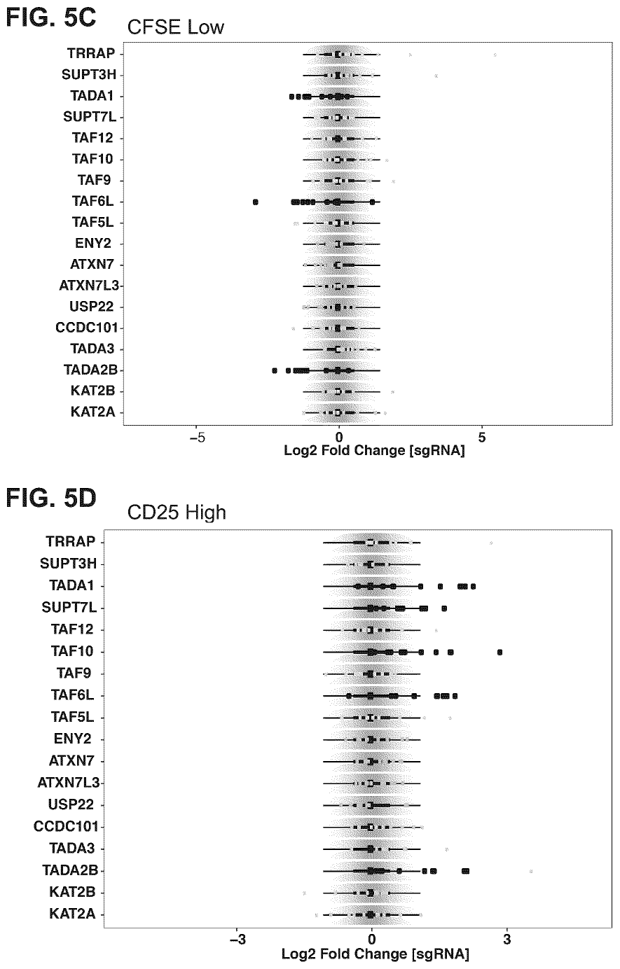

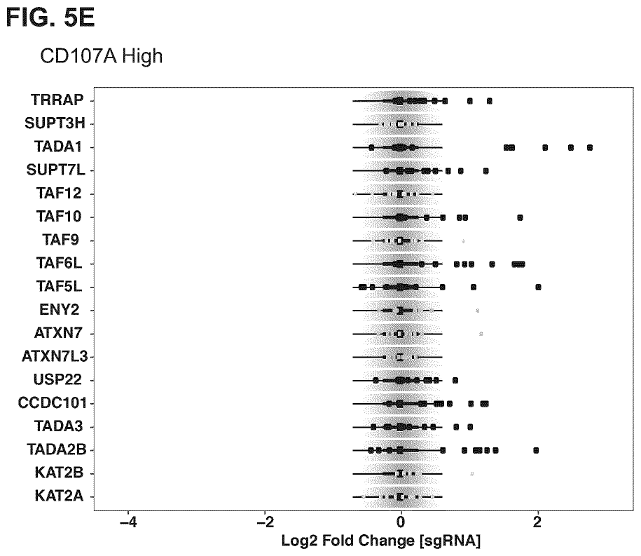

In addition to T cell activation, the present inventors sought to identify positive and negative regulators of CD8+ T cell cytolytic activity (FIG. 3). A key pathway used by CD8+ T cell to kill their target cells is based on granzyme-mediated lethal delivery. CD8+ T cells deliver granules containing Granzyme B and perforin (pore-forming glycoprotein) at the immunological synapses between the T cell and target cell, activating caspases and downstream pro-apoptotic pathways, thus resulting in DNA fragmentation and rapid loss of membrane integrity [24]. Due to its importance in T cell cytolytic activity, FACS sorting Granzyme B high and Granzyme B low population after TCR stimulation was initially tested. However, due to the fixation process during intracellular staining, good quality genomic DNA yields and purity from the sorted populations could not be retained. As a surrogate of cytolytic activity, the inventors stained TCR stimulated cells with CD107a, a cell surface marker expressed during active degranulation [25]. A schematic of this CRISPR screen timeline is shown in FIG. 3A. Cells were FACS sorted into two populations; actively degranulating cells (CD107a high), and non-degranulating cells (CD107a low). With reference to the results shown in FIGS. 3B-3H: As observed with the CFSE CRISPR screen, sgRNAs targeting established regulators of T cell signaling were identified, including CD3D, LCK, CD247 and CD3G. Interestingly, one exception was IRF4, which showed an important role in CD8+ T cell proliferation and activation, but restricted degranulation. The inventors validated IRF4 individual CRISPR knockout with Cas9 ribonucleoproteins (RNPs) and observed an increase in Granzyme B expression after IRF4 knockout. sgRNAs enriched in actively degranulating T cells (CD107a high) were then investigated. Surprisingly, components of the SAGA (Spt-Ada-Gcn5-acetyltransferase) complex were again identified, including TADA2B and CCDC101 (HAT module), TADA1, TAF5L, TAF6L, TAF10, and SUPT7L (core structure module), and TRRAP (TF binding module) among the negative regulators of CD8+ T cell degranulation. The screen was validated by individually knocking out components of the SAGA complex with Cas9 ribonucleoproteins (RNPs) and an approximately 2-fold increase in CD107a expression was observed. In addition, Gene Ontology analysis revealed significant over-representation of gene targets in histone acetyltransferase and SAGA complex in T cells with high CD107a expression. Taken together, these data demonstrate an unappreciated role of the SAGA complex in modulating CD8+ T cell cytolytic activity.

### Example 4

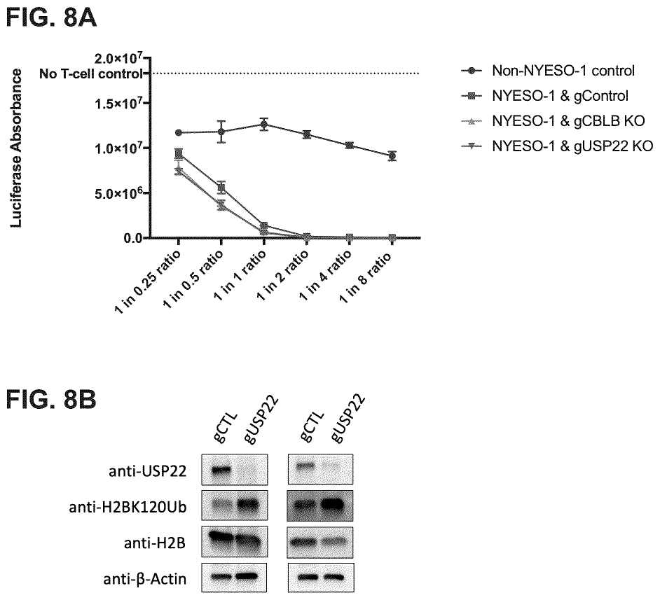

A) Target cell killing by NY-ESO-1-specific primary CD8+ T cells with USP22 gene knockout (SAGA target) and CBLB gene knockout (positive control). With reference to the results shown in FIG. 8A: To demonstrate biological importance, luciferase expressing target cells (NY-ESO-1+ melanoma cell line A375) were incubated with increasing proportions of control wild-type CD8+ T cells or NY-ESO-1-specific CD8+ T cells with CBLB gene knockout (positive control) or USP22 gene knockout (SAGA target). Luminescence was measured to determine the level of A375-target cell killing (N=3, single biological sample, mean±SEM). Knockout of USP22 was as effective at target cell killing as the positive control.

B) Functional validation of USP22 gene knockout in primary CD8+ T cells. With reference to FIG. 8B, western blotting was performed to validate gene knockout of USP22 (N=2, two different biological samples). Gene knockout of USP22 resulted in increased ubiquitination of histone H2B at lysine residue 120.

### REFERENCES

1 Papai, G. et al. (2020) Structure of SAGA and mechanism of TBP deposition on gene promoters. Nature 2011 471:7337 577, 711-716
2 Helmlinger, D. and Tora, L. (2017) Sharing the SAGA. Trends Biochem. Sci. 42, 850-861
3 Basehoar, A. D. et al. (2004) Identification and distinct regulation of yeast TATA box-containing genes. Cell 116, 699-709
4 Huisinga, K. L. and Pugh, B. F. (2004) A genome-wide housekeeping role for TFIID and a highly regulated stress-related role for SAGA in Saccharomyces cerevisiae. Mol. Cell 13, 573-585
5 Baptista, T. et al. (2018) SAGA Is a General Cofactor for RNA Polymerase II Transcription. Mol. Cell 70, 1163-1164
6 Uzhachenko, R. V. and Shanker, A. (2019) CD8+ T Lymphocyte and NK Cell Network: Circuitry in the Cytotoxic Domain of Immunity. Front Immunol 10, 1906
7 Cantrell, D. A. (2003) Regulation and function of serine kinase networks in lymphocytes. Current Opinion in Immunology 15, 294-298
8 Acuto, O. and Michel, F. (2003) CD28-mediated co-stimulation: a quantitative support for TCR signalling. Nature Reviews Immunology 3, 939-951
9 Seidel, J. A. et al. (2018) Anti-PD-1 and Anti-CTLA-4 Therapies in Cancer: Mechanisms of Action, Efficacy, and Limitations. Front. Oncol. 8, 86
10 Chen, L. and Flies, D. B. (2013) Molecular mechanisms of T cell co-stimulation and co-inhibition. Nature Reviews Immunology 13, 227-242
11 Galon, J. et al. (2014) Towards the introduction of the “Immunoscore” in the classification of malignant tumours. J. Pathol. 232, 199-209
12 Zhang, Y. et al. (2020) USP22 controls iNKT immunity through MED1 suppression of histone H2A monoubiquitination. J. Exp. Med. 217, 924
13 Loo, C.-S. et al. (2020) A genome-wide CRISPR screen reveals a role for the BRD9-containing non-canonical BAF complex in regulatory T cells. bioRxiv 9, 2095
14 Cortez, J. T. et al. (2020) CRISPR Screen in Regulatory T Cells Reveals Ubiquitination Modulators of Foxp3. bioRxiv 11, 555
15 Fire, A. et al (1998) Potent and specific genetic interference by double-stranded RNA in Caenorhabditis elegans. Nature, 19:391(6669), 806-11
16 Elbashir S. M. et al. (2001) RNA interference is mediated by 21- and 22-nucleotides RNAs. Genes Dev. 15; 15(2): 188-200
17 Konermann S. et al. (2018) Transcriptome Engineering with RNA-Targeting Type VI-D CRISPR Effectors. Cell 173, 665-676
18 Choi B. D. et al. (2019) CRISPR-Cas9 disruption of PD-1 enhances activity of universal EGFRvIll CAR T cell in a preclinical model of human glioblastoma. J Immunother Cancer. 14; 7(1):304
19 Henriksson J. et al. (2019) Genome-wide CRISPR Screens in T Helper Cells Reveal Pervasive Crosstalk between Activation and Differentiation. Cell 176(4), 882-896
20 Shrifrut E. et al. (2018) Genome-wide CRISPR Screens in Primary Human T Cells Reveal Key Regulators of Immune Function. Cell 175(7), 1958-1971
21 Su S. et al. (2015), CRISPR-Cas9 mediated efficient PD-1 disruption of human primary T cells for adoptive therapy. J Immunother Cancer. 3(Suppl 2): P53
22 Roth T. L. et al. (2018) Reprogramming human T cell function and specificity with non-viral genome targeting. Nature 559, 405-409
23 Stadtmauer E. A. et al. (2020) CRISPR-engineered T Cell in patients with refractory cancer. Science 367(6481)
24 Salti, S. M. et al. (2011) Granzyme B regulates antiviral CD8+ T cell responses. J. Immunol. 187, 6301-6309
25 Alter, G. et al. (2004) CD107a as a functional marker for the identification of natural killer cell activity. J. Immunol. Methods 294, 15-22

What is claimed is:

1. A method for increasing T cell effector function in a T cell population, the method comprising inhibiting the expression or function of the SAGA (Spt-Ada-Gcn5-acetyltransferase) gene regulation complex in T cells of the T cell population.

2. A method for increasing T cell effector function in a T cell population, the method comprising inhibiting one or more genetic subunits of the SAGA (Spt-Ada-Gcn5-acetyltransferase) gene regulation complex in T cells of the T cell population.

3. The method of claim 2, wherein the one or more genetic subunits are selected from ADA2B, CCDC101, TADA1, TAF5L, TAF6L, TAF10, SUPT7L, and TRRAP.

4. A method for increasing T cell effector function in a subject, the method comprising: contacting a T cell population with a composition comprising an inhibitor of the SAGA gene regulation complex ex vivo; and administering a therapeutically effective amount of the T cell population to the subject.

5. The method of claim 4, wherein the subject is a patient diagnosed with cancer.

6. The method of claim 5, wherein the method further comprises administering a cancer therapy to the subject.

7. The method of claim 6, wherein the cancer therapy is an immunotherapy.

8. The method of claim 7, comprising administering to the subject an immune checkpoint inhibitor.

9. The method of any one of claims 1 to 8, wherein the T cells are activated CD8⁺ T cells.

10. The method of any one of claims 1 to 9, wherein the method reduces the activity of the SAGA gene regulation complex in the T cells by at least 70%, at least 80%, at least 90% or at least 99%, relative to a population of T cells wherein the SAGA gene regulation complex is uninhibited.

11. The method of any one of claims 1 to 10, wherein the T cell expresses a Chimeric Antigen Receptor (CAR).

12. The method of any one of claims 1 to 11, wherein the inhibitor is a small molecule.

13. The method of any one of claims 1 to 11, wherein the inhibitor comprises a nucleic acid capable of hybridizing with a nucleic acid encoding a genetic subunit of the SAGA gene regulation complex to inhibit the expression of the subunit.

14. The method of any one of claims 1 to 11, wherein the inhibitor comprises a Cas9 protein or a polynucleotide encoding said Cas9 protein and a CRISPR-cas system guide RNA polynucleotide.

15. A modified T cell that expresses a Chimeric Antigen Receptor (CAR) and wherein the expression or function of one or more of the subunits of the SAGA (Spt-Ada-Gcn5-acetyltransferase) gene regulation complex is inhibited.

16. A population of cells comprising a plurality of the modified T cells of claim 15.

17. A population of cells for use in a method of cancer treatment comprising a population of T cells, wherein one or more genetic subunits of the SAGA (Spt-Ada-Gcn5-acetyltransferase) gene regulation complex are inhibited in the T cell population.

18. The population of cells of any one of claim 16 or 17, wherein the T cells are activated CD8⁺ T cells.

19. The population of cells of any one of claims 16 to 18, wherein the one or more genetic subunits are selected from ADA2B, CCDC101, TADA1, TAF5L, TAF6L, TAF10, SUPT7L, and TRRAP.

20. The population of cells of any one of claims 16 to 19, wherein the activity of the SAGA gene regulation complex in the T cells is inhibited by at least 70%, at least 80%, at least 90% or at least 99%.

21. The population of any one of claims 16 to 20, wherein the inhibitor comprises a nucleic acid capable of hybridizing with a nucleic acid encoding a genetic subunit of the SAGA gene regulation complex to inhibit the expression of the subunit or a Cas9 protein or a polynucleotide encoding said Cas9 protein and a CRISPR-cas9 system guide RNA polynucleotide.
